(19) United States
(12) Patent Application Publication
(10) Pub. No.: US 2015/0018386 A1
(43) Pub. Date: Jan. 15, 2015

(54) AMORPHOUS FORM OF APIXABAN, PROCESS OF PREPARATION AND COMPOSITIONS THEREOF

(71) Applicant: CADILA HEALTHCARE LIMITED, Ahmedabad, Gujarat (IN)

(72) Inventors: Shriprakash Dhar Dwivedi, Ahmedabad (IN); Kamlesh Kumar Singh, Ahmedabad (IN); Jitendra Maganbhai Gajera, Ahmedabad (IN); Nitin Tandon, Ahmedabad (IN); Digambar Ware, Ahmedabad (IN)

(21) Appl. No.: 14/383,521

(22) PCT Filed: Mar. 5, 2013
(86) PCT No.: PCT/IN2013/000131

§ 371 Date: Sep. 5, 2014

(30) Foreign Application Priority Data
Mar. 6, 2012 (IN) 592/MUM/2012

(51) Int. Cl. A61K 31/4545

(52) U.S. Cl. CPC A61K 31/4545
USPC 514/303; 546/120; 428/402

(57) ABSTRACT

The present invention discloses an amorphous form of apixaban, process of preparation and compositions thereof.

### FIELD OF THE INVENTION

The field of the invention relates to an amorphous form of apixaban. In particular, the invention relates to a process for the preparation of an amorphous form of apixaban. The invention also relates to pharmaceutical compositions comprising therapeutically effective amount of an amorphous form of apixaban for oral administration as an antithrombotic agent.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

“Apixaban” is chemically known as 4,5,6,7-tetrahydro-1-(4-methoxyphenyl)-7-oxo-6-[4-(2-oxo-1-piperidinyl)phenyl]-1H-pyrazolo[3,4-c]pyridine-3-carboxamide (CAS name) or 1-(4-methoxyphenyl)-7-oxo-6-[4-(2-oxo-1-piperidinyl) phenyl]-4,5,6,7-tetrahydro-1H-pyrazolo [3,4-c]pyridine-3-carboxamide (IUPAC name) of Formula (I).

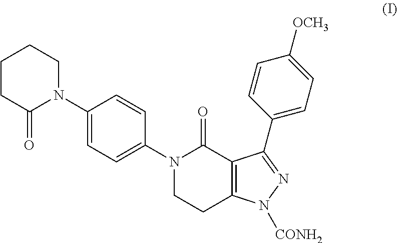 [SMILES: COc1ccc(-c2nn(C(N)=O)c3c2C(=O)N(c2ccc(N4CCCCC4=O)cc2)CC3)cc1]

U.S. Pat. No. 6,967,208 (based on U.S. application Ser. No. 10/245,122) discloses Apixaban, which is herein incorporated by reference in its entirety, has utility as a Factor Xa inhibitor, and is being developed for oral administration in a variety of indications that require the use of an antithrombotic agent.

U.S. Pat. Nos. 7,005,435 B2, 6,989,391 B2, 6,995,172 B2, 7,338,963 B2, 7,371,761 B2, 7,531,535 B2, 7,691,846 B2 and 7,960,411 B2 discloses various analogues compounds of Apixaban. All the patents are incorporated herein by reference in their entirety.

International (PCT) publication No. WO 2003/049681 A2 and its corresponding U.S. Pat. Nos. U.S. 6,919,451 B2 and U.S. 7,153,960 B2 discloses process for the preparation of apixaban and other pyrazole-pyridine derivatives.

International (PCT) publication No. WO 2007/001385 A2 and its corresponding U.S. Pat. Nos. 7,396,932 B2 (the US '932 B2) discloses alternative process for the preparation of apixaban and other pyrazole-pyridine derivatives. The US '932 B2 discloses crystalline form N-1 and form H2-2 of apixaban alongwith the unit cell data thereof.

U.S. Patent Application Publication No. 2007/0203178 A1 discloses crystalline solvates of apixaban viz. dimethyl formamide solvate DMF-5 and formamide solvate Form FA-2 of apixaban characterized by unit cell parameters.

International (PCT) publication No. WO 2011/0106478 A2 (BMS) discloses a composition comprising crystalline apixaban particles having a mean particle size equal to or less than about 89 μm and a pharmaceutically acceptable diluent or carrier.

Crystalline solids normally require a significant amount of energy for dissolution due to their highly organized, lattice like structures. For example, the energy required for a drug molecule to escape from a crystal is more than from an amorphous or a non-crystalline form. It is known that the amorphous forms in a number of drugs exhibit different dissolution characteristics and in some cases different bioavailability patterns compared to the crystalline form (Econno T., Chem. Pharm. Bull., 1990; 38: 2003-2007). For some therapeutic indications, one bioavailability pattern may be favored over another. An amorphous form of Rosuvastatin Calcium, Rabeprazole sodium are some of the examples of one amorphous drug exhibiting much higher bioavailability than the crystalline forms, which leads to the selection of the amorphous form as the final drug substance for pharmaceutical dosage from development. Additionally, the aqueous solubility of cyrstalline atorvastatin calcium is lower than its amorphous form, which may result in the difference in their in vivo bioavailability. Therefore, it is desirable to have amorphous forms of drugs with high purity to meet the needs of regulatory agencies and also highly reproducible processes for their preparation.

In view of the above, it is therefore, desirable to provide an efficient, more economical, less hazardous and eco-friendly process for the preparation of amorphous form of apixaban. The amorphous form provided herein is atleast stable under ordinary stability conditions with respect to purity, storage and is free flowing powder.

### SUMMARY OF THE INVENTION

In one general aspect, there is provided an amorphous solid, dispersion that includes apixaban and a polymer.

In another general aspect, there is provided an amorphous form of apixaban of Formula (I)

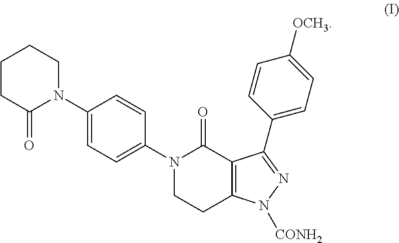 [SMILES: COc1ccc(-c2nn(C(N)=O)c3c2C(=O)N(c2ccc(N4CCCCC4=O)cc2)CC3)cc1]

In another general aspect, there is provided an amorphous form of apixaban of Formula (I) having water content from about 0.5% to about 5% wt/wt.

In another general aspect, there is provided a process for preparation of an amorphous form of apixaban, which includes one or more of the following steps:

a) providing a solution of apixaban in one or more of suitable solvent or mixture thereof; and

b) obtaining an amorphous form of apixaban by removal of solvent.

In another general aspect, there is provided a stable amorphous form of apixaban, which is atleast stable during storage and drying.

In another general aspect, the stable amorphous apixaban, is stored under nitrogen atmosphere and packed in a double polythene bag tied with a thread, keeping primary packing containing amorphous apixaban inside a black color polyethylene bag containing oxygen busters and sealing it, placing above the double polyethylene bag inside a triple laminated bag optionally containing oxygen busters and sealing it, and placing the sealed triple laminated bag inside a closed high density polyethylene (HDPE) container and storing in controlled environment chamber at about 25° C. and/or 40° C.

In another general aspect, there is provided an amorphous apixaban characterized by X-ray powder diffraction as depicted in FIG. 1.

In another general aspect, there is provided an amorphous apixaban having particle size distributions wherein the 10th volume percentile particle size (D10) is less than about 50 μm, the 50th volume percentile particle size (D50) is less than about 200 μm, or the 90th volume percentile particle size (D90) is less than about 400 μm, or any combination thereof.

In another general aspect, there is provided a process for the preparation of amorphous form of apixaban, which comprises:

(a) suspending apixaban in one or more of suitable organic solvent;

(b) removing the organic solvent, and;

(c) obtaining apixaban in amorphous form.

In another general aspect, there is provided a composition of an amorphous form of apixaban having at least one polymer, as well as methods for production of such compositions from a solvent-based medium.

In another general aspect, there is provided an amorphous form of apixaban of Formula (I) having an chiral purity of greater than about 95%, or greater than about 98%, or greater than about 99%, or greater than about 99.5%, or greater than about 99.8%, or greater than about 99.9%, as determined using high performance liquid chromatography (HPLC).

In another general aspect, there is provided a pharmaceutical composition comprising therapeutically effective amount of an amorphous form of apixaban together with one or more pharmaceutically acceptable carriers, excipients or diluents.

In another general aspect, there is provided a pharmaceutical composition comprising therapeutically effective amount of storage stable amorphous form of apixaban together with one or more pharmaceutically acceptable carriers, excipients or diluents.

In another general aspect, there is provided pharmaceutical composition comprising a stabilized composition of an amorphous form of apixaban together with one or more pharmaceutically acceptable carrier, optionally with one or more pharmaceutically acceptable excipients.

### BRIEF DESCRIPTION OF THE ACCOMPANYING DRAWINGS

FIG. 1. Shows the X-ray diffractogram (XRD) of the amorphous form of apixaban as per example-3.

FIG. 2. Shows the X-ray diffractogram (XRD) of the amorphous form of apixaban as per example-4.

FIG. 3. Shows the X-ray diffractogram (XRD) of the amorphous form of apixaban as per example-5

FIG. 4. Shows the X-ray diffractogram (XRD) of the amorphous form of apixaban as per example-6.

### DETAILED DESCRIPTION OF THE INVENTION

The above and other objects of the present invention are achieved by the process of the present invention, which leads to greater stability of amorphous apixaban. The invention provides a process for preparing amorphous form of apixaban in a suitable organic solvent.

Optionally, the solution, prior to any solids formation, can be filtered to remove any undissolved solids, solid impurities and the like prior to removal of the solvent. Any filtration system and filtration techniques known in the art can be used.

As used herein, the terms “suspending”, “slurrying” and “triturating” are interchangeable, and refer to a process carried out in a heterogeneous mixture where complete dissolution does not occur. Also, heating the suspension or slurry can result in a homogenous mixture where complete or partial dissolution occurs at an elevated temperature or ambient temperature.

All ranges recited herein include the endpoints, including those that recite a range “between” two values. Terms such as “about”, “generally”, “substantially,” and the like are to be construed as modifying a term or value such that it is not an absolute. Such terms will be defined by the circumstances and the terms that they modify as those terms are understood by those skill in the art. This includes, at very least, the degree of expected experimental error, technique error and instrument error for a given technique used to measure a value.

As used herein, the term “stable apixaban” includes either: amorphous apixaban that after exposure to a relative humidity of 75% at 40° C., for a period of at least three months contains less than about 0.5% (wt/wt) total impurities and doesn't change to crystalline form and has water content less than 5% wt/wt.

As used herein, the term “stable apixaban” includes either: amorphous apixaban that after exposure to a relative humidity of 60% at 25° C., for a period of at least three months contains less than about 0.5% (wt/wt) total impurities and doesn't change to crystalline form and has water content less than 5% wt/wt.

As used herein, the term “solid dispersion” means any solid composition having at least two components. In certain embodiments, a solid dispersion as disclosed herein includes an active ingredient apixaban dispersed among atleast one other component, for example a polymer.

The term “immobilize” as used herein with reference to the immobilization of the active compound i.e. apixaban in the polymer matrix, means that molecules of the active compound interact with molecules of the polymer in such a way that the molecules of the apixaban are held in the aforementioned matrix and prevented from crystal nucleation due to lack of mobility.

“Suitable solvent” means a single or a combination of two or more solvents.

In one general aspect, there is provided a composition comprising an amorphous form of apixaban. In particular, the composition is a solid dispersion that includes apixaban and a polymer.

In general, the polymer may be a non-ionic polymer or an ionic polymer. The polymer comprises of hydroxypropylmethyl cellulose acetate succinate, hydroxypropylmethyl cellulose, methacrylic acid copolymers, polyvinylpyrrolidone (PVP) and the like. In particular, PVP of different grades like K-15, K-30, K-60, K-90 and K-120 may be used for the preparation of amorphous composition. More particular, hydroxypropylmethyl cellulose acetate succinate and PVP K-30 may be used.

In some embodiments, the apixaban of Formula (I) may be dispersed within a matrix formed by a polymer in its solid state such that it is immobilized in its amorphous form. The polymer may prevent intramolecular hydrogen bonding or weak dispersion forces between two or more drug molecules of apixaban. The solid dispersion provides for a large surface area, thus further allowing for improved dissolution and bioavailability of apixaban.

In some embodiments, the ratio of the amount of weight of apixaban within the solid dispersion to the amount by weight of the polymer therein is from about 1:1 to about 1:10. The composition of apixaban with polymer, preferably PVP K-30 or HPMC-AC may be prepared by using about 1:1 to about 1:10 polymers with respect to apixaban. The usage of higher molar amount of polymer increases the amorphous character of the drug substance.

In another general aspect there is provide a process for the preparation of composition of amorphous apixaban having at least one polymer, the process comprises mixing apixaban with a polymer in a suitable organic solvent and obtaining amorphous composition of apixaban by removal of solvent.

The compound apixaban and a polymer (for example HPMC-AC or PVP K-30) may be dissolved in a suitable organic solvent having a low boiling point, e.g. methanol, ethanol, isopropanol, acetone, ethyl acetate and the like. The amorphous solid dispersion may be obtained by removal of solvent (for example by spray drying, lyophilization, flash evaporation, vacuum distillation and the like) thereby leaving the amorphous solid dispersion precipitated in a matrix formed by the polymer.

In another general aspect, there is provided an amorphous form of apixaban of Formula (I).

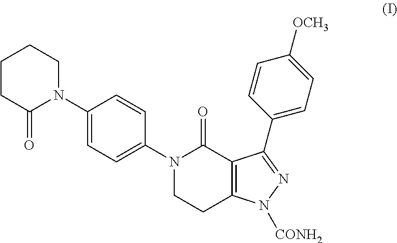 [SMILES: COc1ccc(-c2nn(C(N)=O)c3c2C(=O)N(c2ccc(N4CCCCC4=O)cc2)CC3)cc1]

In another general aspect, there is provided a process for the preparation of amorphous form of apixaban or a salt thereof without simultaneous formation of crystalline forms or which will enable the conversion of crystalline forms into the amorphous from.

In another general aspect, there is provided amorphous form of apixaban of Formula (I) having water content from about 0.5% to about 5% wt/wt.

In another general embodiment, there is provided a process for preparation an amorphous form of apixaban, which includes one or more of the following steps:

a) providing a solution of apixaban in one or more of suitable solvent or mixture thereof; and

b) obtaining an amorphous form of apixaban by removal of solvent.

Step a) involves providing a solution of apixaban or a salt thereof in one or more of solvent or mixture thereof.

The solution for step a) can be obtained by the known methods that includes:

(i) direct use of a reaction mixture containing apixaban that is obtained in the course of its synthesis; or

(ii) dissolving apixaban in one or more of suitable solvent or mixture thereof.

Suitable solvents that may be used in step a) include but are not limited to one or more of alcohols such as methanol, ethanol, isopropanol, 2-propanol, 1-butanol, t-butyl alcohol, and the like; ketones such as acetone, butanone, methyl isobutyl ketone, and the like; esters such as ethyl acetate, isopropyl acetate, t-butyl acetate, isobutyl acetate, chlorinated hydrocarbons such as methylene dichloride, ethylene dichloride, chlorobenzene, and the like, nitriles like acetonitrile, and polar aprotic solvents like N,N-dimethylformamide, N,N-dimethylacetamide, N-methylpyrrolidone, dimethylsulfoxide, and mixtures thereof.

Step b) involves isolation of an amorphous form of apixaban from the solution of step a). The isolation may be affected by removing the solvent. Suitable techniques which may be used for the removal of solvent include using a rotational distillation device such as a Buchi Rotavapor, spray drying, agitated thin film drying (“ATFD”), freeze drying (lyophilization), and the like or any other suitable technique.

Alternatively, isolation can be effected by addition of suitable antisolvent to the solution obtain in step a), optionally by concentrating the solution obtained in step a). Suitable anti-solvents that may be used can be selected from one or more of hydrocarbons like hexanes, n-heptane, n-pentane, cyclohexane, methylcyclohexane and the like; aromatic hydrocarbons like toluene, xylene, ethylbenzene and the like; ethers like diethyl ether, diisopropyl ether, t-butyl methyl ether, dibutyl ether, tetrahydrofuran, 1,4-dioxane, 2-methoxyethanol and the like or water.

In one preferred aspect, there is provided spray drying a solution of apixaban that involves the spray drying of feed stock, which is prepared as discussed below, wherein any crystalline form of apixaban may be used. The feedstock is dozed into the spray-drying instrument JISL Mini Spray-drier LSD-48 and spray drying is carried out under the following parameters.

| Sr. No | Parameters | Conditions | |
|---|---|---|---|
| a) | Feed pump | 10-50 | rpm |
| b) | Inlet temperature | 35°-80° | C. |
| c) | Outlet temperature | 30°-60° | C. |
| d) | Aspirator rate | 1000-1500 | rpm |
| e) | Vacuum for conveying | 30-120 | mm of Hg |
| | the dry product | | |
| f) | Hot air supply | 2-4 | Kg/cm² |
| g) | Atomizer Speed: | 40,000-100,000 | rpm |

In the present invention, feed stock of apixaban is conveniently prepared by dissolving any known forms or wet cake of apixaban in the solvent selected from the group of solvents e.g. acetone, C₁₋₄ alcohol, C₂₋₆ acetate, acetonitrile, methylene dichloride, water or mixture thereof. In particular, methanol, ethanol, acetone, ethyl acetate, methylene dichloride, water-methanol or water-ethanol, water-acetone are suitable solvent used or such solvents that evaporate easily to afford dry product, most particularly acetone, methanol, ethanol, ethyl acetate or mixtures thereof.

In one general aspect, there is provided stable amorphous form of apixaban or a salt thereof of Formula (I)

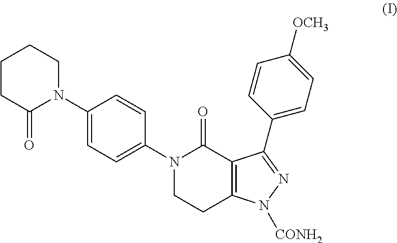 [SMILES: COc1ccc(-c2nn(C(N)=O)c3c2C(=O)N(c2ccc(N4CCCCC4=O)cc2)CC3)cc1]

The invention provides stable amorphous form of apixaban or a salt thereof of Formula (I) having water content from about 0.5% to about 5% wt/wt which is free flowing and does not develop crystallinity under normal stability conditions for atleast about 3 months.

According to another general aspect, any form of apixaban can be spray dried by dissolving or suspending or slurring in suitable solvent or solvent-water system to get amorphous form.

In the present invention feedstock of apixaban in solvent or aqueous solvent system is spray-dried. Thus obtain spray-dried compound is in amorphous form, this fact is again confirmed by the X-ray powder diffractogram of spray-dried apixaban.

In another general aspect, the present invention provides a process for manufacturing amorphous form of apixaban, which comprises:

(a) suspending apixaban in one or more of suitable organic solvent;

(b) removing the organic solvent, and;

(c) obtaining apixaban in amorphous form.

The suspension of apixaban may be a clear solution with homogenous mixture or a suspension or slurry with a heterogeneous mixture in suitable organic solvent selected from the group consisting of ketone, C₁₋₄ alcohol, C₂₋₆ acetate, nitrile, halogenated hydrocarbon, polar aprotic solvents, or mixture thereof. In particular, acetone, methanol, ethanol, ethyl acetate, butyl acetate, isopropyl acetate, acetonitrile, methylene dichloride, water-methanol or water-ethanol, water-acetone, dimethylformamide, dimethylacetamide, dimethylsulfoxide, N-methylpyrrolidone and the like.

In a specific preferred embodiment of the invention, weighed quantity of apixaban may be dissolved in 2-10 volumes of chosen solvent, preferably 4-5 volumes solvent at 25° C. to 30° C. The content may be stirred for 30 minutes at 25° C. to 30° C. The content may be filtered through Hyflosupercell, and filtrate is spray dried under following conditions. The obtained powder may be further dried at 40° C. for 12-16 hours under vacuum to afford stable amorphous form of apixaban or its salt.

| Sr. No | Parameters | Conditions | |
|---|---|---|---|
| a) | Feed pump | 10-50 | rpm |
| b) | Inlet temperature | 35°-80° | C. |
| c) | Outlet temperature | 30°-60° | C. |
| d) | Aspirator rate | 1000-1500 | rpm |
| e) | Vacuum for conveying | 50-120 | mm of Hg |
| | the dry product | | |
| f) | Hot air supply | 2-4 | Kg/cm² |
| g) | Atomizer Speed: | 40,000-100,000 | rpm |

In a preferred feature, the feedstock for spray drying is either a clear solution or in dispersion form.

In another preferred feature, the spray drying of apixaban may be performed by a) maintaining the feed rate of the feed stock at 50-250 ml/hr, preferably 100-200 ml/hr; b) maintaining the inlet temperature in the range of 35° C.-80° C., preferably, 50° C.-70° C.; c) maintaining the aspirator rate between 1000-1500 rpm, preferably 1200-1400rpm; d) maintaining the outlet temperature in the range of 30° C. to 60° C., preferably, 40° C. to 50° C.; e) maintaining air flow at 2-4 Kg/cm, preferably 2 Kg/cm; f) maintaining Atomizer speed between 20,000-100,000 rpm, preferably, 40,000-50,000 rpm, and; g) maintaining the vacuum at 30-120 mm of Hg, preferably 50-80 mm of Hg.

In another general aspect, there is provided an amorphous form of apixaban, substantially free from of residual organic solvents.

In further aspect, the stable amorphous apixaban, can be stored under nitrogen atmosphere and packed in a double polythene bag tied with a thread, keeping primary packing containing amorphous apixaban inside a black color polyethylene bag containing oxygen busters and sealing it, placing above the double polyethylene bag inside a triple laminated bag optionally containing oxygen busters and sealing it, and placing the sealed triple laminated bag inside a closed high density polyethylene (HDPE) container and storing in controlled environment chamber at about 25° C. and/or 40° C.

In another general aspect, there is provided an amorphous form of apixaban of Formula (I) having an chiral purity of greater than about 95%, or greater than about 98%, or greater than about 99%, or greater than about 99.5%, or greater than about 99.8%, or greater than about 99.9%, as determined using high performance liquid chromatography (HPLC).

In another general aspect, there is provided a pharmaceutical composition comprising an amorphous form of apixaban substantially free residual solvents as measured by GC together with one or more pharmaceutically acceptable carriers, excipients or diluents.

In another general aspect, there is provided a pharmaceutical composition comprising an amorphous form of apixaban substantially free from crystalline forms together with one or more pharmaceutically acceptable carriers, excipients or diluents.

In another general aspect, there is provided pharmaceutical composition comprising therapeutically effective amount of an amorphous apixaban having at least one polymer together one or more of pharmaceutically acceptable carriers, excipients or diluents

In another general aspect, there is provided amorphous form of apixaban having particle size distributions wherein the 10th volume percentile particle size (D10) is less than about 50 μm, the 50th volume percentile particle size (D50) is less than about 200 μm, or the 90th volume percentile particle size (D90) is less than about 400 μm, or any combination thereof.

Powder X-ray Diffraction of amorphous apixaban can be obtained under following conditions.

(i) Characterization by Powder X-ray Diffraction

The X-ray powder diffraction spectrum was measured under the following experimental conditions:

Instrument: X-Ray Diffractometer, D/Max-2200/PC Make: Rigaku, Japan.

X-Ray: Cu/40 kv/40 mA

Diverging: 10

Scattering Slit: 10

Receiving Slit: 0.15 mm

Monochromator RS: 0.8 mm

Counter: Scintillation Counter

Scan Mode: Continuous

Scan Speed: 3.0000/Min

Sampling Width: 0.020

Scan Axes: Two Theta/Theta

Scan Range: 2.0000 to 40.0000

Theta Offset: 0.0000

According to another aspect, apixaban to be used as the starting material may be prepared by the known methods reported in the prior i.e. by using the process as per U.S. Pat. Nos. 6,967,208 B2 and 7,396,932 B2 or CN 101967145 A, which is incorporated herein as reference.

The invention also encompasses pharmaceutical compositions comprising apixaban of the invention. As used herein, the term “pharmaceutical compositions” includes pharmaceutical formulations like tablets, pills, powders, liquids, suspensions, emulsions, granules, capsules, suppositories, or injection preparations.

Pharmaceutical compositions containing the apixaban of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

In another general aspect, there is provided a pharmaceutical composition comprising therapeutically effective amount of amorphous form of apixaban together with one or more pharmaceutically acceptable excipients.

In another general aspect, there is provided a pharmaceutical composition comprising therapeutically effective amount of storage stable amorphous form of apixaban substantially free from crystalline form together with one or more pharmaceutically acceptable carriers, excipients or diluents.

In another general aspect, there is provided a pharmaceutical composition comprising a stabilized amorphous solid dispersion of apixaban together with one or more pharmaceutically acceptable carrier, optionally with one or more pharmaceutically acceptable excipients.

Having described the invention with reference to certain preferred embodiments, other embodiments will become apparent to one skilled in the art from consideration of the specification.

### EXAMPLE-1

100 mg (0.217 m mol) of apixaban and 15 mL methanol were taken in round bottom flask at 25-30° C. The reaction mixture was heated at 45-50° C. to obtain clear solution. 100 mg of PVP-K30 polymer was added and stirred at 45-50° C. for 2 hours. The reaction mixture was distilled under vacuum at 60-65° C. The product was dried under vacuum at 55-60° C. to obtain 120 mg amorphous apixaban.

### EXAMPLE-2

100 mg (0.217 mmol) of apixaban and 15 mL methanol were taken in round bottom flask at 25-30° C. The reaction mixture was heated at 45-50° C. to obtain clear solution. 200 mg of PVP-K30 polymer was added and stirred at 45-50° C. for 2 hours. The reaction mixture was distilled under vacuum at 60-65° C. The product was dried under vacuum at 55-60° C. to obtain 155 mg amorphous apixaban.

### EXAMPLE-3

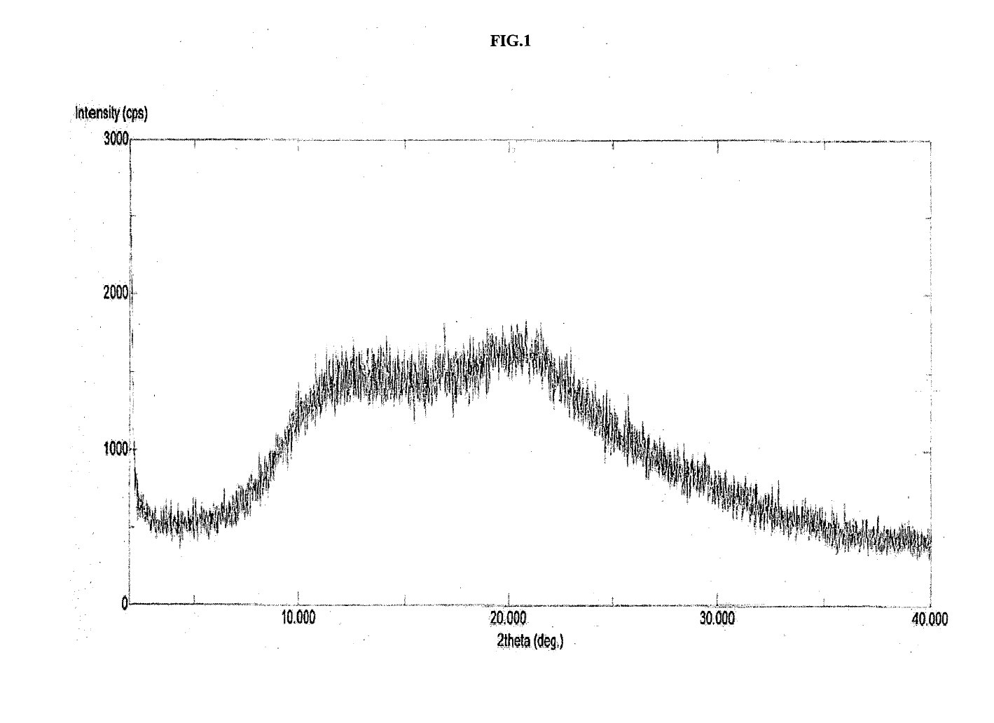

50 mg (0.108 mmol) of apixaban and 10 mL methanol were taken in round bottom flask at 25-30° C. The reaction mixture was heated at 45-50° C. to obtain clear solution. 200 mg of PVP-K30 polymer was added and stirred at 45-50° C. for 2 hours. The reaction mixture was distilled under vacuum at 60-65° C. The product was dried under vacuum at 55-60° C. to obtain 125 mg amorphous apixaban. (XRD: FIG. 1)

### EXAMPLE-4

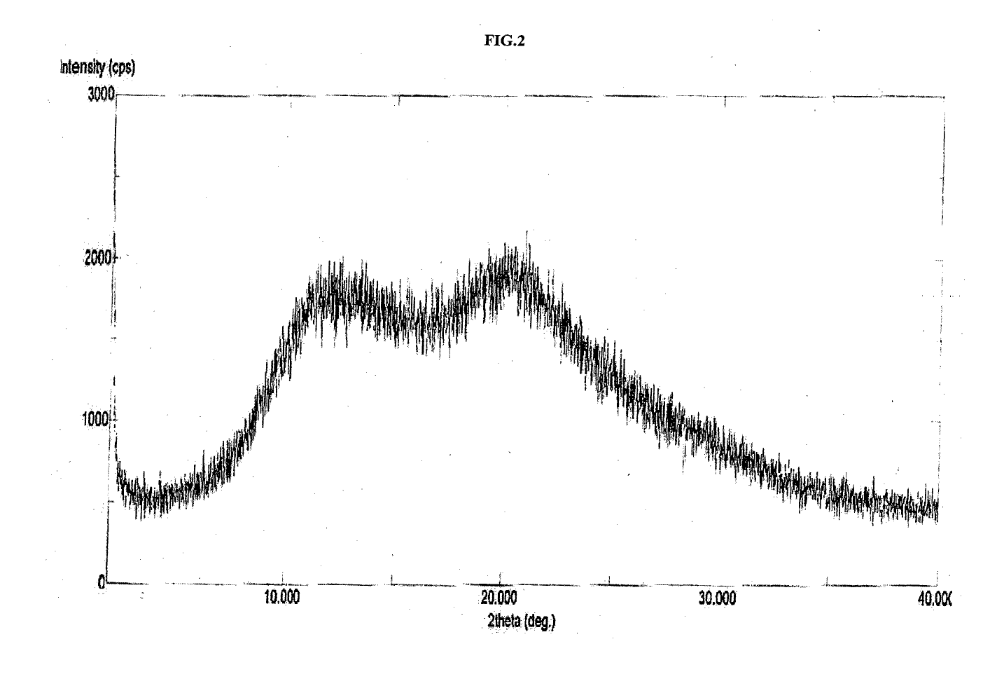

40 mg (0.087 mmol) of apixaban and 10 mL methanol were taken in round bottom flask at 25-30° C. The reaction mixture was heated at 45-50° C. to obtain clear solution. 320 mg of PVP-K30 polymer was added and stirred at 45-50° C. for 2 hours. The reaction mixture was distilled under vacuum at 60-65° C. The product was dried under vacuum at 55-60° C. to obtain 145 mg amorphous apixaban. (XRD: FIG. 2).

### EXAMPLE-5

50 mg (0.108 mmol) of apixaban and 65 mL methanol were taken in round bottom flask at 25-30° C. The reaction mixture was heated at 45-50° C. to obtain clear solution.

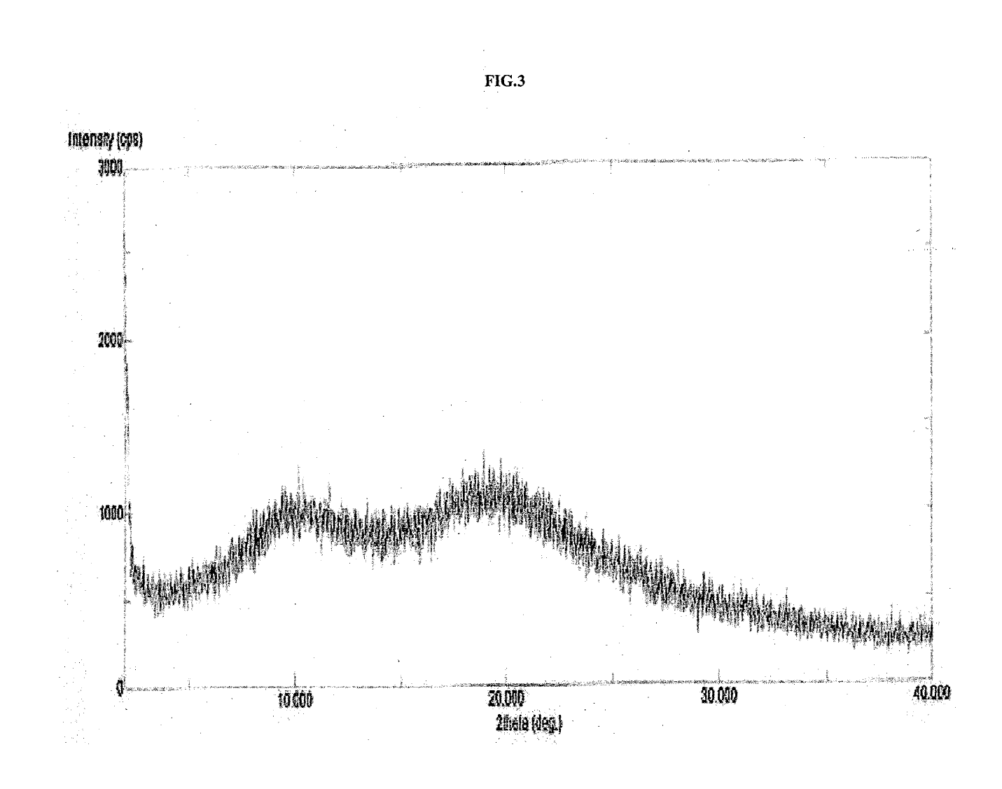

200 mg of HPMC-AS polymer was added and stirred at 45-50° C. for 2 hours. The reaction mixture was distilled under vacuum at 60-65° C. The product was dried under vacuum at 55-60° C. to obtain 125 mg amorphous apixaban. (XRD: FIG. 3).

### EXAMPLE-6

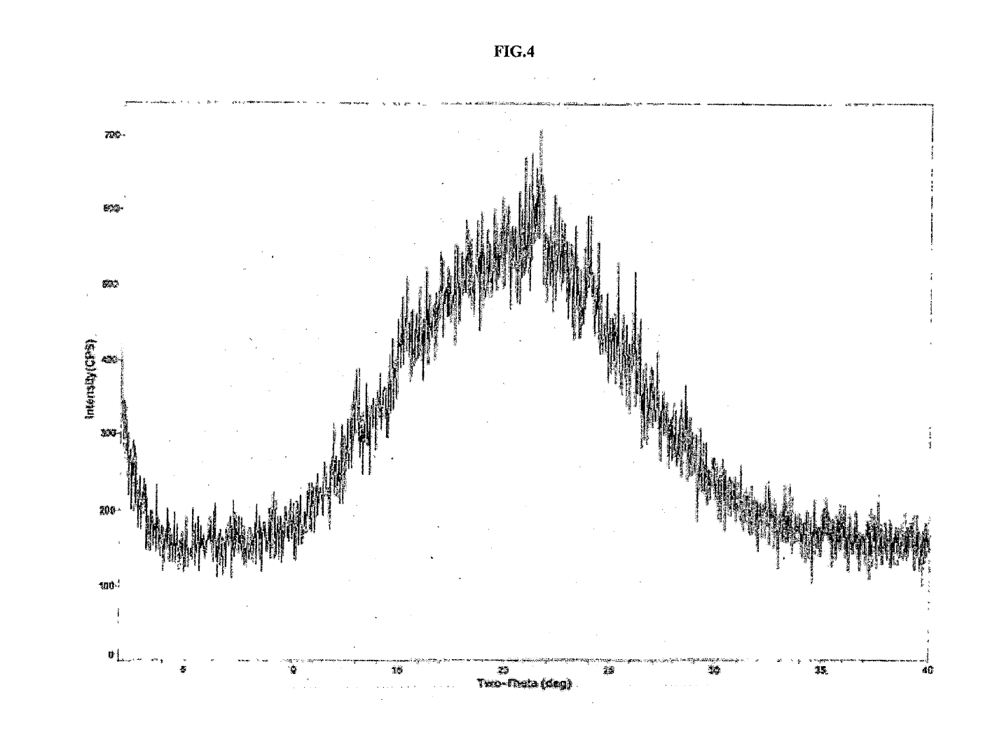

25.0 g of apixaban was dissolved in 250.0 mL of methanol at 25° C. to 30° C. The content was stirred for 30 minutes at 25° C. to 30° C. To this, 1.0 g charcoal was added and stirred for 30 minutes at 80° C. The content was filtered through Hyflosupercell, and the Hyflosupercel pad is washed with 50 mL methanol. The filtrate was concentrated under vacuum below 45° C. till 100 mL methanol remains. 50 mL methanol was added and stirred at 80° C. to get clear solution, followed by spray drying in JISL Mini spray drier LSD-48 under the below conditions. The product was collected from cyclone and was further dried at 40° C.±5° C. under vacuum for 16 hours to get 22.0 g of amorphous apixaban. The obtained solid was amorphous as is shown by the X-ray diffraction pattern given in FIG. 4.

| Sr. No | Parameters | Conditions | |
|---|---|---|---|
| a) | Feed pump | 30 | rpm |
| b) | Inlet temperature | 60° | C. |
| c) | Outlet temperature | 40° | C. |
| d) | Aspirator rate | 1300 | rpm |
| e) | Vacuum for conveying | 80 | mm of Hg |
| | the dry product | | |
| h) | Hot air supply | 2 | Kg/cm² |

The spray-dried apixaban is amorphous in nature. The obtained product contains residual solvent well within ICH limit.

### EXAMPLE-7

25.0 g of apixaban was dissolved in 200.0 mL of acetone at 25° C. to 30° C. The content was stirred for 30 minutes at 65° C. To this, 2.0 g charcoal was added and stirred for 30 minutes at 65° C. The content was filtered through Hyflosupercell, and the Hyflosupercell pad was washed with 50.0 mL acetone. The filtrate was concentrated under vacuum below 45° C. till 100 mL acetone remains. 50 mL acetone was added and stirred to get clear solution, followed by spray drying in JISL Mini spray drier LSD-48 under the below conditions. The product was collected from cyclone and was further dried at 40° C.±5° C. under vacuum for 16 hours to get 21 g of amorphous apixaban.

| Sr. No | Parameters | Conditions | |
|---|---|---|---|
| a) | Feed pump | 30 | rpm |
| b) | Inlet temperature | 60° | C. |
| c) | Outlet temperature | 40° | C. |
| d) | Aspirator rate | 1300 | rpm |
| e) | Vacuum for conveying | 80 | mm of Hg |
| | the dry product | | |
| h) | Hot air supply | 2 | Kg/cm² |

The spray-dried apixaban is amorphous in nature. The obtained product contains residual solvent well within ICH limit, chiral purity >99% and water content by less than 5% wt/wt.

### EXAMPLE-8

25.0 g of apixaban was dissolved in 250.0 mL of ethyl acetate at 25° C. to 30° C. The content was stirred for 30 minutes at 65° C. To this, 2.0 g charcoal was added and stirred for 30 minutes at 65° C. The content was filtered through Hyflosupercell, and the Hyflosupercell pad was washed with 50.0 mL ethyl acetate. The filtrate was concentrated under vacuum below 45° C. till 100 mL ethyl acetate remains. 50 mL ethyl acetate was added and stirred to get clear solution, followed by spray drying in JISL Mini spray drier LSD-48 under the below conditions. The product was collected from cyclone and was further dried at 40° C.±5° C. under vacuum for 16 hours to get 18 g of amorphous apixaban.

| Sr. No | Parameters | Conditions | |
|---|---|---|---|
| a) | Feed pump | 30 | rpm |
| b) | Inlet temperature | 60° | C. |
| c) | Outlet temperature | 40° | C. |
| d) | Aspirator rate | 1300 | rpm |
| e) | Vacuum for conveying | 80 | mm of Hg |
| | the dry product | | |
| h) | Hot air supply | 2 | Kg/cm² |

The spray-dried apixaban is amorphous in nature. The obtained product contains residual solvent well within ICH limit, chiral purity >99% and water content by less than 5% wt/wt.

### EXAMPLE 9

25 g of apixaban was dissolved in 200 ml ethyl acetate by heating at 75° C. to 80° C. The slightly turbid solution was filtered through hyflow bed at 75° C. to 80° C. The filtrate was cooled to 25° C. and added to methyl tert-butyl ether (200 mL). The reaction mixture was stirred for 2 hours. The reaction mixture was filtered and wet-cake was washed with methyl tert-butyl ether (100 mL). The product was dried in hot air oven for 12 hours to get 17.0 g amorphous apixaban.

### EXAMPLE 10

25 g of apixaban and methanol (200 mL) were stirred in RBF for 30 minutes. The reaction mixture was distilled at 45° C. to 50° C. under vacuum to obtain dry product. The filtrate was distilled under vacuum till dry powder obtained at 45° C. to 50° C. The solid was cooled to 25° C. and cyclohexane (200 mL) was added to the filtrate and stirred for 30 min. The reaction mixture was filtered and wet-cake was washed with cyclohexane (200 mL). The product was dried in hot air oven for 12 hours to get 15.0 g amorphous apixaban.

While the present invention has been described in terms of its specific embodiments, certain modifications and equivalents will be apparent to those skilled in the art and are intended to be included within the scope of the present invention.

We claim:

1. A composition comprising an amorphous form of apixaban.

2. The composition according to claim 1 is a solid dispersion that includes apixaban and a polymer.

3. The composition according to claim 2 wherein the ratio of the amount of weight of apixaban within the solid dispersion to the amount by weight of the polymer therein is from about 1:1 to about 1:10.

4. The composition according to claim 2 wherein the polymer comprises hydroxypropyl-methyl cellulose acetate succinate, hydroxypropylmethyl cellulose, methacrylic acid copolymers, polyvinylpyrrolidone (PVP) and the like.

5. The composition according to claim 4 wherein polyvinylpyrrolidone (PVP) comprises of different grades like K-15, K-30, K-60, K-90 and K-120.

6. A process for the preparation of composition comprising amorphous form of apixaban having at least one polymer, the process comprises mixing apixaban with a polymer in a suitable organic solvent and obtaining composition comprising form of apixaban by removal of solvent.

7. The process according to claim 6 wherein suitable organic solvent comprises low boiling organic solvent.

8. The process according to claim 7 wherein low boiling solvent comprises of methanol, ethanol, isopropanol, acetate, ethyl acetate and the like.

9. An amorphous form of apixaban of Formula (I).

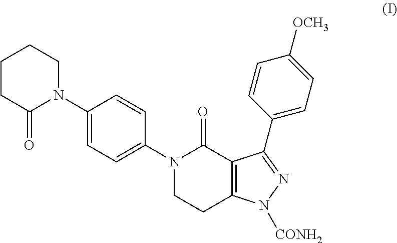 [SMILES: COc1ccc(-c2nn(C(N)=O)c3c2C(=O)N(c2ccc(N4CCCCC4=O)cc2)CC3)cc1]

10. The amorphous form of apixaban according to claim 9, wherein the water content is from about 0.5% to about 5% wt/wt.

11. A stable amorphous form, of apixaban.

12. The stable amorphous form of apixaban according to claim 11 after exposure to a relative humidity of 75% at 40° C., for a period of at least three months contains less than about 0.5% (wt/wt) total impurities and doesn't change to crystalline form and has water content less than 5% wt/wt.

13. The stable amorphous form of apixaban according to claim 11 after exposure to a relative humidity of 60% at 25° C., for a period of at least three months contains less than about 0.5% (wt/wt) total impurities and doesn't change to crystalline form and has water content less than 5% wt/wt.

14. A process for preparing an amorphous form of apixaban, which includes one or more of the following steps:
(a) providing a solution of apixaban in one or more of suitable solvent or mixture thereof; and
(b) obtaining an amorphous form of apixaban by removal of solvent.

15. The process according to claim 14 wherein suitable solvent comprises one or more of methanol, ethanol, isopropanol, 2-propanol, 1-butanol, t-butyl alcohol, and the like; ketones such as acetone, butanone, methyl isobutyl ketone, and the like; esters such as ethyl acetate, isopropyl acetate, t-butyl acetate, isobutyl acetate, chlorinated hydrocarbons such as methylene dichloride, ethylene dichloride, chlorobenzene, and the like, nitriles like acetonitrile, and polar aprotic solvents like N,N-dimethylformamide, N,N-dimethylacetamide, N-methylpyrrolidone, dimethyl sulfoxide, and mixtures thereof.

16. The process according to claim 14 wherein the amorphous apixaban is obtained by removal of solvent.

17. The process according to claim 16 wherein solvent may be removed by suitable techniques like a rotational distillation device such as a Buchi Rotavapor, spray drying, agitated thin film drying (“ATFD”), freeze drying (lyophilization), and the like or any other suitable technique.

18. The process according to claim 14 further comprises obtaining amorphous apixaban by addition of suitable anti-solvent.

19. The process according to claim 18 wherein suitable anti-solvent comprises one or more of hydrocarbons like hexanes, n-heptane, n-pentane, cyclohexane, methylcyclohexane and the like; aromatic hydrocarbons like toluene, xylene, ethylbenzene and the like; ethers like diethyl ether, diisopropyl ether, t-butyl methyl ether, dibutyl ether, tetrahydrofuran, 1,4-dioxane, 2-methoxyethanol and the like or water.

20. A process for the preparation of amorphous form of apixaban, which comprises:
(a) suspending apixaban in one or more of suitable organic solvent;
(b) removing the organic solvent, and;
(c) obtaining amorphous form of apixaban.

21. The process according to claim 20, wherein suitable organic solvent comprises one or more of acetone, methanol, ethanol, ethyl acetate, butyl acetate, isopropyl acetate, acetonitrile, methylene dichloride, water-methanol or water-ethanol, water-acetone, dimethylformamide, dimethylacetamide, dimethylsulfoxide, N-methylpyrrolidone and the like.

22. An amorphous apixaban substantially free from residual solvents.

23. A pharmaceutical composition comprising therapeutically effective amount of amorphous apixaban having at least one polymer together one or more of pharmaceutically acceptable carriers, excipients or diluents.

24. The composition according to claim 23, wherein water-soluble polymer comprises of polyvinylpyrrolidone (PVP) or hydroxypropylmethyl cellulose acetate succinate (HPMC-AC).

25. A pharmaceutical composition comprising therapeutically effective amount of amorphous form of apixaban together with one or more of pharmaceutically acceptable carriers, excipients or diluents.

26. A pharmaceutical compositions comprising amorphous form of apixaban substantially free from crystalline forms.

27. A pharmaceutical composition comprising therapeutically effective amount of storage stable amorphous form of apixaban substantially free from crystalline form together with one or more pharmaceutically acceptable carriers, excipients or diluents.

28. A pharmaceutical compositions comprising amorphous form of apixaban substantially free from crystalline forms together with one or more pharmaceutically acceptable carriers, excipients or diluents.

29. An amorphous form of apixaban having particle size distributions wherein the 10th volume percentile particle size (D10) is less than about 50 μm, the 50th volume percentile particle size (D50) is less than about 200 μm, or the 90th volume percentile particle size (D90) is less than about 400 μm, or any combination thereof.
